(19) United States
(12) Patent Application Publication
(10) Pub. No.: US 2007/0072229 A1
(43) Pub. Date: Mar. 29, 2007

(54) METHOD FOR ISOLATION, AMPLIFICATION AND QUANTITATION OF RIBONUCLEIC ACID

(75) Inventors: Carolyn Kay Bialozynski, Wauwatosa, WI (US); Christopher Michael Rosenau, Milwaukee, WI (US); T.S. Rama Subramanian, Mequon, WI (US)

Correspondence Address: WOOD, HERRON & EVANS, LLP, 2700 CAREW TOWER, 441 VINE STREET, CINCINNATI, OH, 45202, US

(73) Assignee: Pierce Milwaukee, L.L.C., Milwaukee, WI (US)

(21) Appl. No.: 11/536,297

(22) Filed: Sep. 28, 2006

Related U.S. Application Data
(60) Provisional application No. 60/721,881, filed on Sep. 28, 2005.

(51) Int. Cl. C12Q 1/68; C12N 1/08

(52) U.S. Cl. 435/6; 435/270

(57) ABSTRACT

A cellular RNA isolation method comprising treating at least one nucleated cell with a composition comprising mild detergents such as non-ionic detergent(s), buffer(s), chelator(s), reducing agent(s), salt(s), and RNase inhibitor(s) to result in a cytoplasmic lysate and intact nucleus, then separating the cytoplasmic lysate containing RNA from the nucleus, and using the cytoplasmic lysate for RNA analysis without further purifying the RNA.

### RELATED APPLICATION

This application claims priority from U.S. application Ser. No. 60/721,881 filed Sep. 28, 2005, which is expressly incorporated by reference herein in its entirety.

### BACKGROUND

Isolation and purification of ribonucleic acid (RNA) is commonly a pre-requisite step for measurement of gene expression levels. Current methods designed for monitoring gene expression in this manner exhibit inherent shortcomings. These methods commonly use reagents that lyse the entire cell, thereby causing the desired spliced and fully functional cytoplasmic RNA to be in a matrix with nuclear debris that can interfere with downstream applications, necessitating a purification process. These methods also do not efficiently retain small RNA species. Given the increasing knowledge of the role of microRNA (miRNA) in gene expression/regulation, retention of all cytoplasmic RNA species is necessary to correlate miRNA expression with mRNA expression.

### BRIEF DESCRIPTION OF THE DRAWINGS

FIG. 1 shows possible outcomes in the presence and absence of cell component compartmentalization.

FIG. 2 shows quantitative reverse transcription-polymerase chain reaction (qRT-PCR) results using β-tubulin intron-exon primers.

FIG. 3 shows qRT-PCR results using β-tubulin intra-exon primers.

FIG. 4 shows direct reverse transcription-polymerase chain reaction (RT-PCR) results from cytoplasmic lysates using intra-exon primers.

FIG. 5 shows direct RT-PCR results from tissue cytoplasmic lysates.

FIG. 6 shows qRT-PCR results from cytoplasmic lysates using two measurement systems.

FIG. 7 shows small interfering RNA (siRNA)-mediated silencing from cytoplasmic lysates.

FIG. 8 shows protein levels from the siRNA-mediated silencing from cytoplasmic lysates of FIG. 7.

FIG. 9 shows detection of microRNA (miRNA) in cytoplasmic lysates.

FIG. 10 shows miRNA recovery in the cytoplasmic fraction.

### DETAILED DESCRIPTION

This application contains at least one drawing executed in color. A Petition under 37 C.F.R. §1.84 requesting acceptance of the color drawings is filed separately on even date herewith. Copies of this patent with color drawing(s) will be provided by the Office upon request and payment of the necessary fee.

A method is disclosed for use of ribonucleic acid (RNA) directly from cytoplasmic lysates, without any purification of the RNA. Cytoplasmic lysates are achieved by selective lysis (“gentle” lysis) of cells, where only the cellular membrane is disrupted. The cytoplasmic lysates are directly assayed or used in downstream applications for measuring or detecting RNA. Nuclear membranes are not lysed and nuclei are removed so that there is no need for a deoxyribonucleic acid (DNA) removal step. The method effectively compartmentalizes and allows access to all the cytoplasmic RNA species without interference by nuclear components. The cytoplasmic fraction of cells is effectively separated from the nuclear components with one reagent mixture in less than about 20 minutes, in about 10 minutes, in about 5 minutes, or in about one minute, in various embodiments. The method allows selective analysis of fully processed RNA. The method encompasses a selective lysis whereby the outer membrane of the cell is lysed without lysing the nuclei using a non-ionic, ionic, or other mild detergent in the presence of other agents, such as a salt, buffer, chelating agent, reducing agent, and RNase inhibitor. Following separation of the cytoplasmic lysate from the nuclei (e.g. by centrifugation or other methods), the lysate, or dilutions of the lysate, is used directly in downstream applications without further purification or DNA removal. Such applications include, but are not limited to, reverse transcription (RT) and polymerase chain reaction (PCR), quantitative and competitive RT-PCR, quantitative reverse transcription PCR (qRT-PCR), ribonuclease protection assays (RPA), Northern blots, primer extension assays, RNA quantitation assays, monitoring siRNA-mediated silencing of gene expression, profiling ribosomal RNA (rRNA) signatures, monitoring micro RNA (miRNA), studying RNA-protein interactions and complexes, simultaneous assay of RNA and protein to correlate the expression level of RNA with protein, direct analysis of proteins via polyacrylamide gels and Western blots, etc., details of each of which are known in the art and/or are found in Sambrook, Fritsch, Maniatis (1989) Molecular Cloning: A Laboratory Manual, Cold Spring Harbor Laboratory Press, the relevant portions of which are expressly incorporated by reference herein.

The method uses a non-ionic and/or a mildly ionic detergent(s) as a mild lysing agent. For example, ionic detergents such as sodium cholate and sodium deoxycholate at concentrations lower than about 0.1% (w/w) can be included. In one embodiment, the detergent is mildly ionic 0.05% (w/w) sodium dodecyl sulfate (SDS) (Sigma-Aldrich, St. Louis Mo.) and non-ionic 0.5% NP-40 (US Biologicals, Swampscott Mass.). In one embodiment, the detergent is mildly ionic 0.05% (w/w) SDS. In one embodiment, the detergent is non-ionic such as Nonidet P40 and may be at a concentration in the range of about 0.5% (w/w) to about 2% (w/w). Other non-ionic detergents, such as Triton X-100 (Sigma-Aldrich, St. Louis Mo.), can also be used either alone or in combinations, and may be at a concentration in the range of about 0.5% (w/w) to about 2% (w/w). In one embodiment, the detergent is 0.5% (w/w) NP-40 with Triton X-100. Detergents that may be used in the inventive method include agents capable of preferentially lysing the cellular outer membrane while leaving the nuclear membrane substantially intact and include non-ionic detergents, examples of which may be used alone or in combination and include, but are not limited to, Brij® family (Sigma-Aldrich, St. Louis Mo.), decaethylene glycol monododecyl ether, N-decanoyl-N-methylglucamine, n-decyl a-D-glucopyranoside, decyl b-D-maltopyranoside, n-dodecanoyl-N-methylglucamide, n-dodecyl a-D-maltoside, heptaethylene glycol monodecyl ether, heptaethylene glycol monotetradecyl ether, n-hexadecyl b-D-maltoside, hexaethylene glycol monododecyl ether, hexaethylene glycol monohexadecyl ether, hexaethylene glycol monooctadecyl ether, hexaethylene glycol monotetradecyl ether, methyl-6-O-(N-heptylcarbamoyl)-a-D-glucopyranoside, nonaethylene glycol monododecyl ether, N-nonanoyl-N-methylglucamine, Nonidet P-40, octaethylene glycol monodecyl ether, octaethylene glycol monohexadecyl ether, octaethylene glycol monooctadecyl ether, octaethylene glycol monotetradecyl ether, octyl-b-D-glucopyranoside, pentaethylene glycol monodecyl ether, pentaethylene glycol monohexadecyl ether, pentaethylene glycol monohexyl ether, pentaethylene glycol monooctadecyl ether, pentaethylene glycol monooctyl ether, polyethylene glycol diglycidyl ether, polyethylene glycol ether, polyoxyethylene 10 tridecyl ether, polyoxyethylene 100 stearate, polyoxyethylene 20 isohexadecyl ether, polyoxyethylene 20 oleyl ether, polyoxyethylene 40 stearate, polyoxyethylene 50 stearate, polyoxyethylene 8 stearate, polyoxyethylene bis(imidazolyl carbonyl), polyoxyethylene 25 propylene glycol stearate, saponin, Span® (Sigma-Aldrich, St. Louis, Mo.), Surfact-Amps family (Pierce Biotechnologies, Rockford Ill.), Tergitol, tetradecyl-b-D-maltoside, tetraethylene glycol monodecyl ether, tetraethylene glycol monododecyl ether, tetraethylene glycol monotetradecyl ether, triethylene glycol monodecyl ether, triethylene glycol monododecyl ether, triethylene glycol monohexadecyl ether, triethylene glycol monooctyl ether, triethylene glycol monotetradecyl ether, Triton® family (Sigma-Aldrich, St. Louis Mo.), TWEEN® family (Sigma-Aldrich, St. Louis Mo.), tyloxapol, tyloxapol, and n-undecyl b-D-glucopyranoside. Zwitterionic detergents such as CHAPS and CHAPSO may be used. In one embodiment, the lysis conditions, particularly the detergent concentration used in lysing a particular biologic sample, may take into account the cholesterol content of that sample.

The lysis reagent also contains a chelator(s), buffer(s), salt(s), reducing agent(s), and RNase inhibitor(s). Examples of these include, but are not limited to, ethylenediaminetetraacetic acid as a chelator (EDTA, Sigma-Aldrich); buffers to maintain pH of about 8 (ranging between pH 7.5 to pH 8.5), such as N-2-hydroxyethylpiperazine-N′-2-ethanesulfonic acid (HEPES), phosphate buffer, and/or Tris-HCl (each available from Sigma-Aldrich); reducing agents such as dithiothreitol (DTT), β-mercaptoethanol, and/or Tris-carboxyethyl-phosphine-HCl (TCEP-HCl) (each available from Sigma-Aldrich); salts such as KCl and/or NaCl (Sigma-Aldrich); and RNase inhibitors such as placental RNase inhibitor or porcine RNase inhibitor (EMD Biosciences, San Diego Calif.), or Protector RNase inhibitor (Roche Applied Science, Indianapolis Ind.).

In one embodiment, the concentration of buffer(s) is about 0.02 M. In one embodiment, the concentration of buffer(s) ranges from about 0.01 M to about 0.05 M. In one embodiment, the concentration of buffer(s) ranges from about 10 mM to about 50 mM. In one embodiment, the concentration of salt(s) is about 0.012 M. In one embodiment, the concentration of salt(s) ranges from about 0.010 M to about 0.015 M. In one embodiment, the concentration of salt(s) ranges from about 0.01 M to about 0.15 M. In one embodiment, the concentration of salt(s) ranges from about 10 mM to about 150 mM. In one embodiment, the concentration of SDS is about 0.05% (w/w). In one embodiment, the concentration of SDS ranges from about 0.01% (w/w) to about 0.05%. In one embodiment, the concentration of NP40 is about 0.5% (w/w). In one embodiment, the concentration of NP 40 ranges from about 0.5% (w/w) to about 2% (w/w). In one embodiment, the concentration of Triton X100 is about 0.5% (w/w). In one embodiment, the concentration of Triton X100 ranges from about 0.25% (w/w) to about 1% (w/w). In one embodiment, the concentration of each of NP40 and SDS is, respectively about 0.5% (w/w) and about 0.05% (w/w). In one embodiment, the concentration of each of NP40 and SDS ranges, respectively between about 0.01% (w/w) to about 0.05% (w/w). In one embodiment, the concentration of chelator(s) is about 0.002 M. In one embodiment, the concentration of chelator(s) ranges from about 0.001 M to about 0.004 M. In one embodiment, the concentration of chelator(s) ranges from about 1 mM to about 4 mM. In one embodiment, the concentration of reducing agent(s) is about 0.001 M. In one embodiment, the concentration of reducing agent(s) ranges from about 0.001 M to about 0.005 M. In one embodiment, the concentration of reducing agent(s) ranges from about 1 mM to about 5 mM. In one embodiment, the concentration of RNase inhibitor(s) ranges from about 0.2 unit/μl to about 0.4 units/μl. In one embodiment, the concentration RNase inhibitor(s) ranges from about 0.03 units/μl to about 0.4 units/μl.

In one embodiment, the lysis reagent contains Tris buffer and/or phosphate buffer and/or HEPES buffer, KCl and/or NaCl, DTT, EDTA, 0.5% (w/w) NP-40 and 0.05% (w/w) SDS. In one embodiment, the lysis reagent contains Tris buffer and/or phosphate buffer and/or HEPES buffer, EDTA, KCl, DTT, 0.5% (w/w) NP-40 and 0.05% (w/w) SDS. In one embodiment, the lysis reagent contains Tris buffer and/or phosphate buffer and/or HEPES buffer, EDTA, NaCl, DTT, 0.5% (w/w) NP-40 and 0.05% SDS. In one embodiment, the lysis reagent contains Tris buffer and/or phosphate buffer and/or HEPES buffer, EDTA, KCl and/or NaCl, TCEP, 0.5% (w/w) NP-40 and 0.05% SDS. In one embodiment, the lysis reagent contains Tris buffer and/or phosphate buffer and/or HEPES buffer, EDTA, KCl and/or NaCl, β-mercaptoethanol, 0.5% (w/w) NP-40, and 0.05% (w/w) SDS. In one embodiment, the lysis reagent contains Tris buffer and/or phosphate buffer and/or HEPES buffer, EDTA, NaCl and/or KCl, TCEP, 0.5% (w/w) NP-40, and 0.05% (w/w) SDS. In one embodiment, the lysis reagent contains Tris buffer and/or phosphate buffer and/or HEPES buffer, EDTA, NaCl and/or KCl, β-mercaptoethanol and/or DTT and/or TCEP, 0.5% (w/w) NP-40, and 0.05% SDS. In one embodiment, the lysis reagent contains Tris buffer and/or phosphate buffer and/or HEPES buffer, EDTA, NaCl and/or KCl, β-mercaptoethanol and/or DTT and/or TCEP, and 0.05% SDS. In one embodiment, the lysis reagent contains Tris buffer and/or phosphate buffer and/or HEPES buffer, EDTA, NaCl and/or KCl, β-mercaptoethanol and/or DTT and/or TCEP, and 0.5% (w/w) NP-40 and 0.5% Triton X-100.

The source of RNA may be any biological material that contains nucleated cells. Thus, tissues, isolated cells, cells in culture, blood, yeast, etc., may be used as known by one skilled in the art. For example, adherent cell lines such as HeLa cells, mouse macrophage cells (e.g., RAW 264.7), and suspension cell lines such as THP-1 cells may be used.

Cells may have been experimentally manipulated prior to processing by the inventive method, to include transfection and exposure to an agent. An example of transfection is introduction of siRNA directed to a gene into a cell, such as a gene encoding glyceraldehyde phosphate dehydrogenase (GAPD(H)) introduced into HeLa cells, using transfection reagents such as siPORT™ NeoFX™ transfection reagent (Ambion).

Tissue samples can be either fresh or frozen, from biopsies or primary cells, and include but are not limited to liver, kidney, brain, muscle, etc. The samples may be directly subjected to the method or further disassociated by mechanical and/or enzymatic disruption prior to lysis.

The biologic sample can also be non-animal nucleated cells, e.g., organisms of the domain Eukarya such as yeast, protozoan, etc.

In one embodiment, the RNase inhibitor is added as a separate component, but at the same time the remaining components (detergent(s), chelator(s), salt(s), buffer(s), and reducing agent(s)) are added to the cells or tissues. In one embodiment, the RNase inhibitor is added as a separate component to the remaining components (detergent(s), chelator(s), salt(s), buffer(s), and reducing agent(s)), and then the mixture is added to the cells or tissues. In one embodiment, the RNase inhibitor is added as a separate component to the cells or tissues, then the remaining components (detergent(s), chelator(s), salt(s), buffer(s), and reducing agent(s)) are added to the mixture of RNase inhibitor/cells or tissues.

The biologic sample is mixed with the lysis reagent under conditions sufficient to result in selective lysis. In one embodiment, the volume of the lysis reagent was about 600 μl for about 1×10⁶ cells or about 0.02 g tissue. In one embodiment, the volume of the lysis reagent ranged from about 300 μl to about 900 μl for about 1×10⁶ cells or about 0.02 g tissue. In one embodiment, a time period in the range of about one min to about fifteen min and a temperature in the range of about 0° C. to about 4° C. is used. The sample in the lysis reagent may be continuously mixed or left to incubate following initial mixing. After lysis, cell debris and nuclei are removed from the lysate, for example, by centrifugation and/or filtration, and the lysate containing RNA is used directly and without further purification in downstream applications. The lysate may also be diluted in buffer appropriate to the downstream application.

In one embodiment, the lysate is diluted in the range of about 1:2 fold to about 1:100 fold for RT-PCR or qRT-PCR reactions. In one embodiment, the lysate is diluted in the range of about 1:00 fold to about 1:100,000 fold for qRT-PCR reactions. As known to one skilled in the art, the dilution may depend upon the cell/tissue type, the particular RNA being measured, etc. For example, lysing 1×10⁶ cells in a 600 microliter volume and then diluting 1 microliter of the lysate about 1:1500 to about 1:15,000 for detection by qRT-PCR would be equivalent to using between 1 to 0.1 cells, or about 5 pg to about 0.5 pg total RNA.

In one embodiment, components of the inventive method are included in a kit. In one embodiment, the RNase inhibitor reagent is segregated or packaged separately from the detergent, buffer, chelator, reducing agent, and salt reagents. In this embodiment, instructions are provided for adding the RNase inhibitor to the other reagents substantially immediately before use. The kit may also contain reagents for RT-PCR and/or qRT-PCR. For example, in one embodiment, the kit contains materials to perform the inventive method along with reagents to perform RT-PCR and/or qRT-PCR (e.g. enzymes, buffers, primers such as random hexamers, oligo dT, and labeled and/or tagged sequence-specific primers and nucleotide derivatives). The kit can also contain reagents necessary for control reactions, such as primers to housekeeping genes (e.g., actin, GAPD(H), tubulin, etc.). In one embodiment, the kit can contain reagents for high throughput purification for miRNA. Non-limiting examples include the following. In one embodiment, a kit for one and two-step lysis-RT-PCR includes lysis reagent(s), RNase inhibitor, a reverse transcriptase such as AMV or MMLV, buffer for RT that contains Tris, MgCl₂ and salt such as KCl and reducing agent such as DTT, oligo T and random hexamer primers with or without modifications, deoxy nucelotides (dATP, dGTP, dCTP, TTP), a DNA polymerase such as Taq, Pfu, etc., buffer for PCR that contains Tris buffer, MgCl₂ and salt such as KCl, and control primers for RT-PCR. In one embodiment, a kit for one-step lysis-qRT-PCR includes lysis reagent(s), RNase inhibitor, reverse transcriptases such as AMV or MMLV, SYBR green reaction one-step buffer that contains Tris, MgCl₂, a salt such as KCl, a reducing agent such as DTT, and dyes such as SYBR green, deoxy nucelotides (dATP, dGTP, dCTP, TTP), and a DNA polymerase (modified or unmodified) such as Taq, Pfu, etc., a reference dye such as ROX, and control primers for RT-PCR. In one embodiment, a kit for two-step lysis-qRT-PCR includes lysis reagent(s), RNase inhibitor, a reverse transcriptase such as AMV or MMLV, buffer for RT that contains Tris, MgCl₂, a salt such as KCl, and a reducing agent such as DTT, oligo T and random hexamer primers with or without modifications, PCR reaction buffer that contains Tris, MgCl₂, a salt such as KCl, and dyes such as SYBR green, deoxy nucelotides (dATP, dGTP, dCTP, TTP), a DNA polymerase (modified or unmodified) such as Taq, Pfu, etc., a reference dye such as ROX, and control primers for RT-PCR. In one embodiment, a kit for cytoplasmic RNA purification includes lysis reagent(s), RNase inhibitor, filter tips/plates to filter cell debris, columns containing a matrix such as glass fiber that binds RNA, buffers containing high salt for binding to columns, and buffers containing low salt for washing and eluting RNA. Other reagents and components are known to one skilled in the art.

Cytoplasmic fractions of cells from culture or from tissues were separated from nuclear components with one reagent in about twenty minutes. The resulting extracts were used directly without any purification step in RT-PCR and qRT-PCR to monitor gene expression, monitor siRNA-mediated gene silencing, etc. Either cultured cells and/or tissues may be in all embodiments. If cultured cells are used, the cells may be adherent or in suspension. If tissues are used, the tissue may be fresh or frozen.

In one embodiment, the method is used for analyzing cell components, such as RNA and protein. Nucleated cells are treated with the disclosed cell lysis reagent, resulting in a cytoplasmic lysate containing RNA and protein, and an intact nucleus. The cytoplasmic lysate is then separated from the nucleus, and the cytoplasmic lysate is used, for example, for RNA analysis without further purifying the RNA, for protein analysis, and/or for RNA-protein analysis. In one embodiment, proteins are analyzed by polyacrylamide gel electrophoresis, Western blots, qualitative assay, and/or quantitative assay. In one embodiment, RNA-protein analysis is by correlating RNA-protein levels, correlating microRNA, messenger RNA, and protein levels, and monitoring RNA-protein interactions.

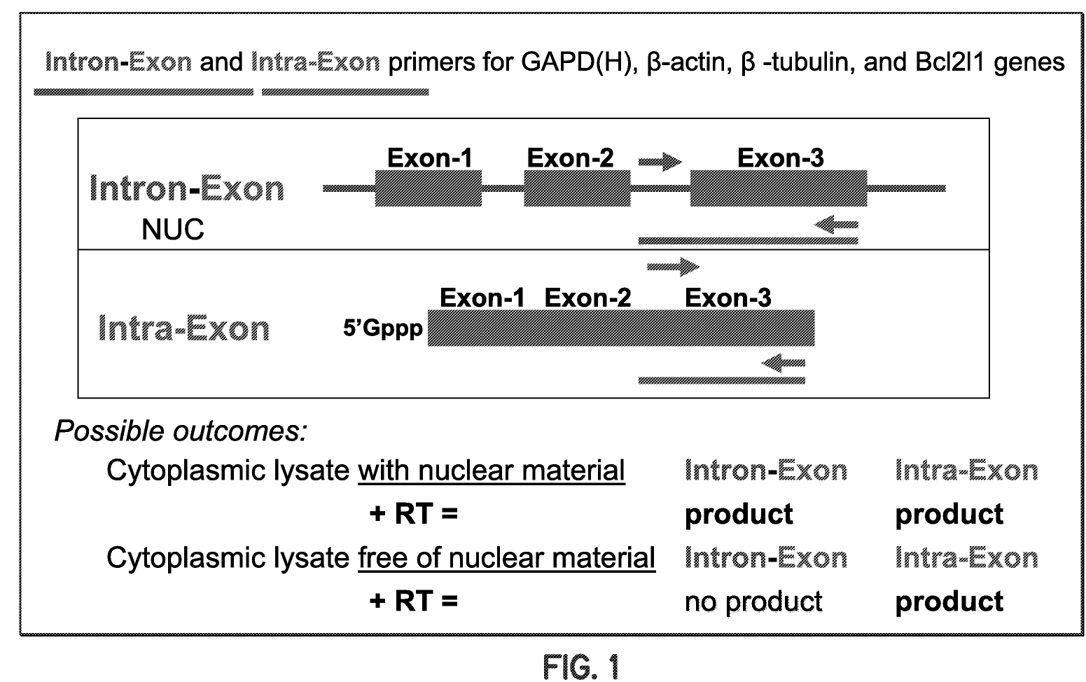

FIG. 1 schematically represents control reactions to verify selective lysis using PCR primers directed to the exon of the gene (intra-exon) or to span the intron/exon junction of a gene (intron-exon), either in the presence or absence of prior RT treatment.

The invention can be further appreciated with respect to the following examples.

### EXAMPLE 1

Cells at a concentration in the range of about 1×10⁶ to 4×10⁶ were re-suspended by mixing in the previously described lysis reagent. The mixture was placed on ice (about 0° C. to about 4° C.) for about 5-10 min (or for about 15 minutes when tissue was used), after which the mixture was centrifuged at about 16,000×g for about 1-5 min at 4° C. The resulting supernatant contained the unpurified cytoplasmic lysate and was added directly and without subsequent purification to the RT-PCR (1:2 to 1:100 dilution) reaction or the qRT-PCR (1:100 to 1:100,000 dilution) reaction.

In one embodiment, the lysis reagent contained 0.05% (w/w) SDS and NP-40 at about 0.5% (w/w), HEPES buffer at about 0.02 M, NaCl at about 0.012 M, EDTA at about 0.002 M, DTT at about 0.001 M, and RNase inhibitor at about 0.3 units/μl to about 0.4 units/μl. In one embodiment, the lysis reagent contained NP-40 at about 0.5% (w/w), HEPES at about 0.02 M, KCl at about 0.012 M, EDTA at about 0.002 M, TCEP-HCl at about 0.001 M, and RNase inhibitor at about 0.2 units/μl to about 0.4 units/μl. In all embodiments, the volume of the lysis reagent was about 600 μl for about 1×10⁶ cells (or about 0.02 g tissue).

Nuclei were then separated from the lysate, e.g. by centrifugation and/or filtrations. In one embodiment, the lysate was then used directly for downstream applications. In another embodiment, the lysate was diluted in a buffer appropriate to the downstream application before use in the downstream application.

For RT-PCR downstream applications, the lysate was added to a reaction mixture for reverse transcription, or alternatively, the RT reaction mixture was added to the lysate. In one embodiment, the RT-PCR reaction was conducted as a one-step process where both RT and PCR were carried out in the same container. In one embodiment, the RT-PCR reaction was conducted as a two-step process where the lysate was first subjected to RT reaction and then an aliquot of the reaction was used in the DNA amplification reaction.

### EXAMPLE 2

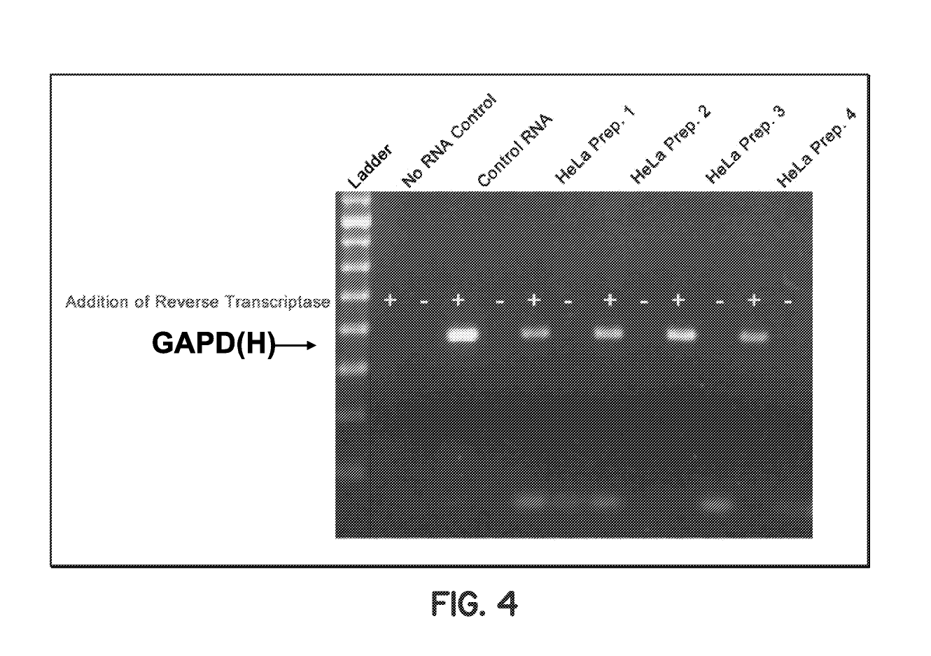

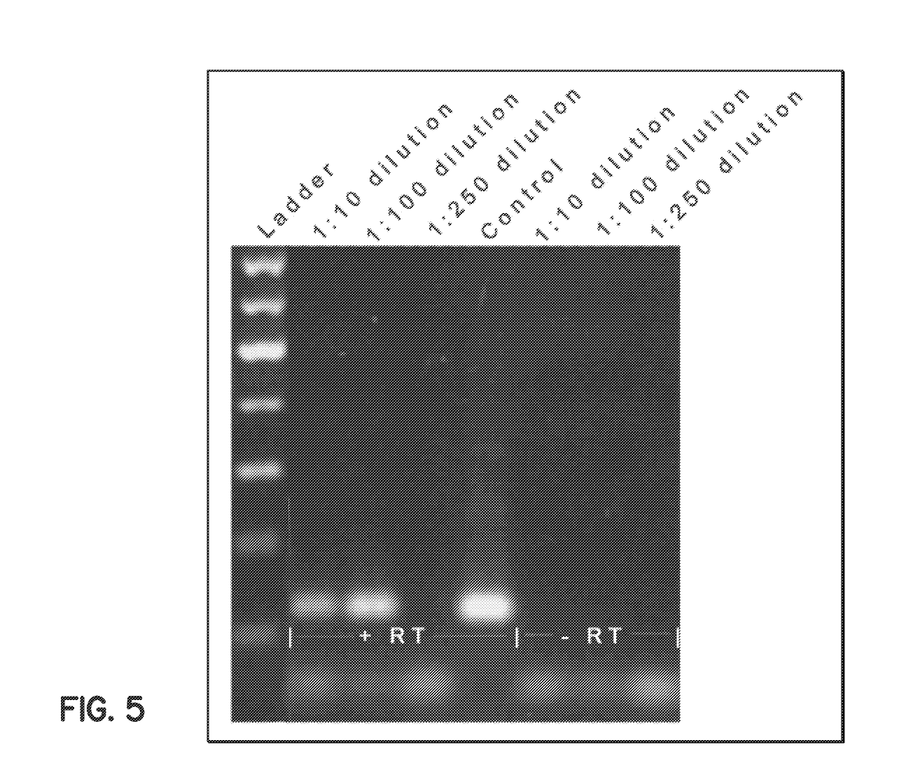

An aliquot of the above RT reaction was used in the DNA amplification reaction (PCR) to generate an amplicon for the gene/RNA of interest using primers specific to the gene/RNA of interest, with results shown in FIGS. 4 and 5.

About 540,000 HeLa cells were trypsinized, centrifuged at about 1300×g for two minutes, and rinsed with cold PBS. Pellets were processed as described. RT-PCR was performed by priming with random hexamers, reverse transcription with MMuLV, and 30 cycle PCR with primers specific to exon 13 of the human GAPD(H) gene. 100 ng of human HeLa cell total RNA (control) or a 1:10 dilution of unpurified cytoplasmic lysate (representing about 1000 cells) was added to each reaction. Products were subject to electrophoresis on a 1% agarose gel and visualized with ethidium bromide. Compartmentalization has also been proven with intra-exon RT-PCR via β-actin amplification in HeLa cells and Bcl2l1 amplification in mouse liver and kidney cells (data not shown). The presence of the amplicon of GAPD(H) in reactions performed in the presence of reverse transcriptase (+), and the absence of any amplified product in reactions done in absence of reverse transcriptase (−), indicate that the cytoplasmic lysate was free of DNA and the amplicon was derived from RNA.

As shown in FIG. 4, in the absence of reverse transcriptase, cytoplasmic lysates did not produce any detectable amplicon. These results confirmed that the lysate was DNA free.

About 0.2 g of fresh mouse kidney tissue was diced, placed in a homogenizer, and mixed with cytoplasmic lysis reagent as described. The samples were placed on ice for 15 minutes, mixed for 10-15 strokes or until pulverized, and centrifuged at about 16,000×g at 4° C. for ten minutes. The supernatant containing unpurified cytoplasmic lysates was collected. RT-PCR was performed by priming with random hexamers, reverse transcription with MMuLV, and 30 cycle PCR with primers specific to exon 2 of the mouse Bcl2l1 gene. 100 ng of human HeLa cell total RNA (control) or dilutions of unpurified cytoplasmic lysates specified above were added to each reaction. Products were subject to electrophoresis on a 1% agarose gel and visualized with ethidium bromide. The presence of the amplicon of GAPD(H) in reactions performed in the presence of reverse transcriptase (+), and the absence of any amplified product in reactions done in absence of reverse transcriptase (−), indicated that the cytoplasmic lysate was free of DNA and the amplicon was derived from RNA

As shown in FIG. 5, cytoplasmic lysates from tissue were used directly in RT-PCR to measure gene expression, with no clean up or DNA removal step needed prior to the RT-PCR reaction.

Alternatively, random or oligo T primers were used to amplify all or a majority of the genes/RNAs present in the sample. The complementary DNA (cDNA) generated can also be used to generate cDNA libraries and probes for detecting gene targets in, for example, gene arrays. Conversely, RT-PCR, Northern blot, RNase protection assay, etc. conducted on the lysate can be used to verify results obtained from gene expression microarrays.

For example, errors in microarray data can arise from a variety of sources, including cross-hybridization, alternative splicing, contamination of clones, mistakes in sequencing, and the fact that hybridization conditions must be ‘one-size-fits-all’ across an array. Other problems include the possibility of an error during the arraying process or the possibility that minor cross-contamination with a clone representing a highly expressed gene will obscure the signal from one of low expression. Therefore, the results for interesting genes are often validated individually by an independent method such as RT-PCR, Northern blot, or RNase protection. Following removal of nuclei and cellular debris, RT and PCR may be conducted in the same container in a one-step operation. The one-step method may include anchoring the primers to the container wall or to beads or another solid support matrix, as known to one skilled in the art.

### EXAMPLE 3

Cells in a multi-well format, such as 24-well, 96-well, 384-well, and 1536-well formats, were subjected to the method of Example 1. In one embodiment, the cells were treated at once. In one embodiment, the cells were treated in a time-based manner, allowing for both the incorporation of many experimental conditions and/or temporal-based determinations. For example, cells in a multiwell format were centrifuged following lysis by the inventive method to pellet cellular debris and nuclei, with the supernatant used directly in assays. Alternatively, the lysate may be filtered to remove particulates (cell debris and nuclei) and the filtrate used in downstream applications. The basic protocol set forth in Example 1 can be applied to high throughput formats to allow, for example, monitoring gene expression over short periods and/or under many experimental conditions (e.g., see FIG. 4). Further, because RNA is not purified but rather is assayed directly from the lysate, any purification-induced bias is eliminated when profiling global gene expression, correlating mRNA to protein levels, or correlating miRNA to mRNA and protein levels.

The lysate generated by the inventive method also exhibited low viscosity, making the resulting sample more amenable to high throughput and/or automated assay systems. The amount of RNA required in downstream applications is often small; therefore the inventive method can be used with small amounts of starting material. Depending on the downstream application to which the lysate is subjected, fewer than 100 cells, fewer than 10 cells, or fewer than 5 cells may be used, lending the method to use in high throughput assays.

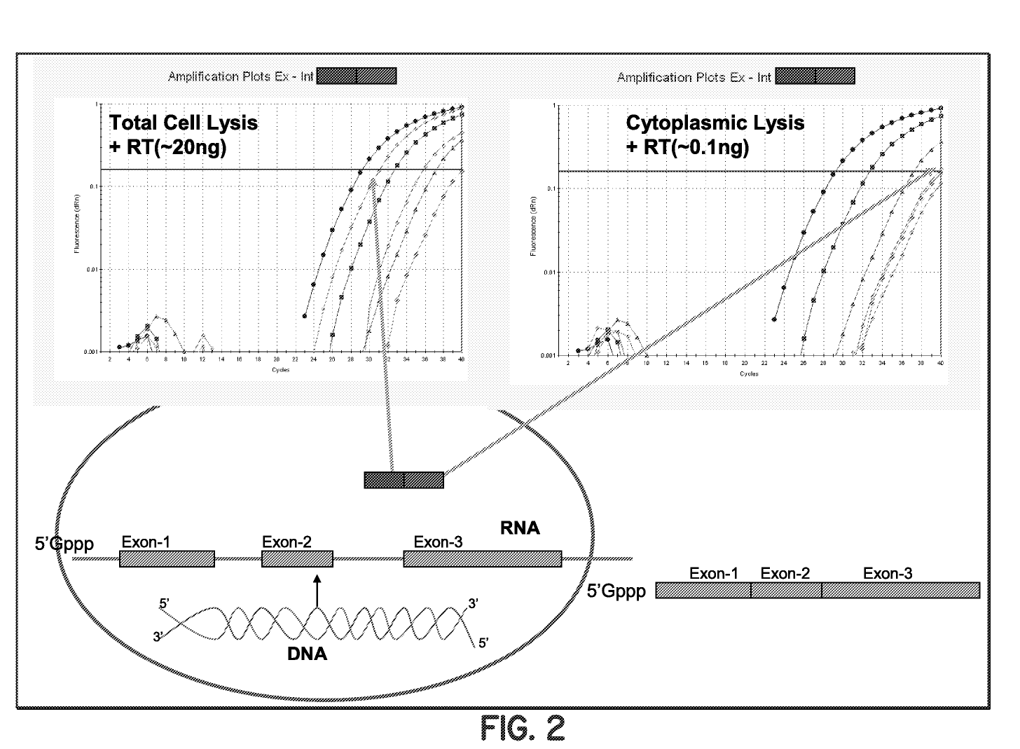

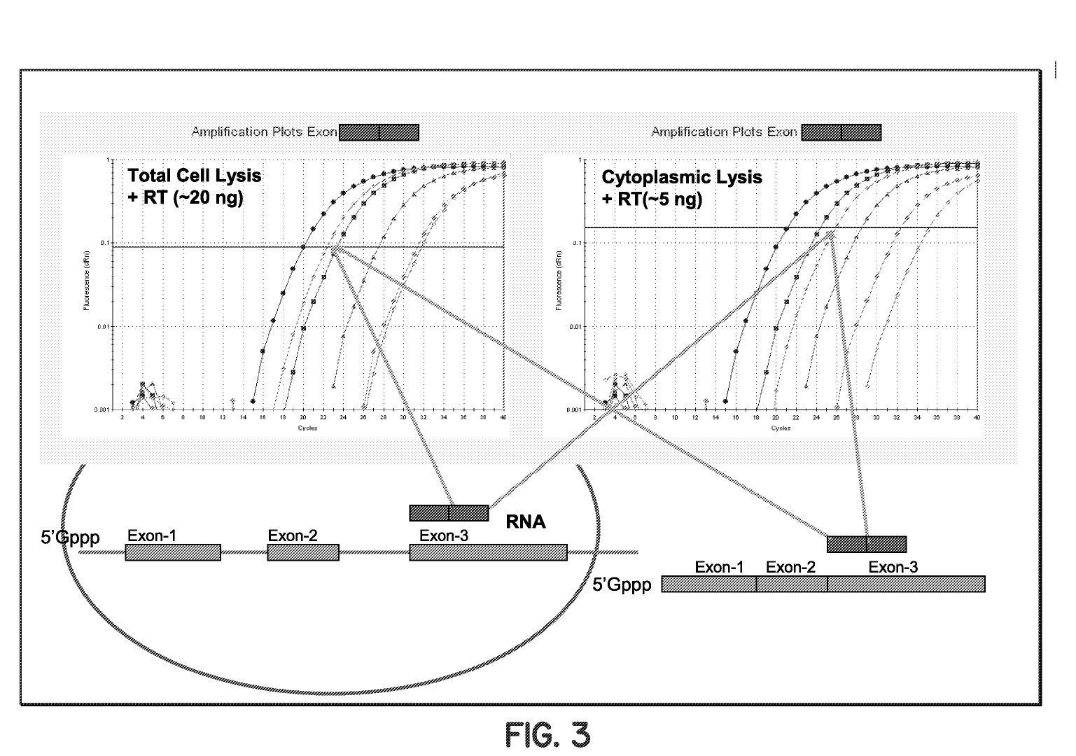

About 2×10⁷ HeLa cells were trypsinized, centrifuged at about 1,000×g for 1 minute, and rinsed with cold PBS. Pellets containing 1×10⁶ cells were processed as described. qRT-PCR was performed by using Brilliant® SYBR® Green qRT-PCR Master Mix, 1-step kit (Stratagene) with primers specific to an intron-exon region of the human beta-tubulin gene (FIG. 2) and primers within an exon (exon-exon) (FIG. 3). Dilutions ranging from 100 ng to 100 pg of human HeLa cell total RNA (standard curve) or a 1:100 dilution of total cell extract or unpurified cytoplasmic lysate (representing about 100 cells) was added to each reaction. Data were collected on a Stratagene Mx3000P™ Real Time PCR System.

For each of FIGS. 2 and 3, each data point in each line represents the amount of amplicon generated (as a measure of fluorescence, along the Y axis) at each PCR cycle (PCR cycle number across the X axis). In FIG. 2, amplification of intron-exon region from total cell lysate (graph on left) generated about 20 ng of amplicon (between 100 ng (red) and 10 ng (blue) of standard RNA), and amplification of intron-exon region from cytoplasmic lysate (graph on right) generated less than 0.1 ng of amplicon, indicating the presence of unprocessed (intron-containing) RNA and/or DNA in total cell lysate, and the reduced amount or absence of unprocessed RNA and DNA in cytoplasmic lysates. In FIG. 3, amplification of intra-exon region from total cell lysates (graph on left) generated about 20 ng of amplicon (between 100 ng (red) and 10 ng (blue) of standard RNA), and amplification of intra-exon region from cytoplasmic lysate (graph on right) generated about 5 ng of amplicon, indicating the presence of RNA in both total cell lysates as well as cytoplasmic lysates. Compartmentalization has also been proven with intron-exon RT-PCR via β-actin and GAPD(H) amplification in HeLa cells and β-actin in THP-1 cells (data not shown).

As shown in FIGS. 2, 3, 6, and 7, fluorescent dyes and/or probes were used to facilitate sample detection, including processing in a high throughput format. For example, qRT-PCR was performed using fluorescently labeled primers, such as Lux™ fluorogenic primers (Invitrogen), DNA intercalating dyes such as SYBR® Green (Molecular Probes, Inc.), or Taqman™ probes (ABI). The increase in fluorescent signal was measured using equipment such as the Mx3000P™ Real Time PCR System (Stratagene) and correlated with the production of the PCR product, and thus was used to quantitate the amount of product made.

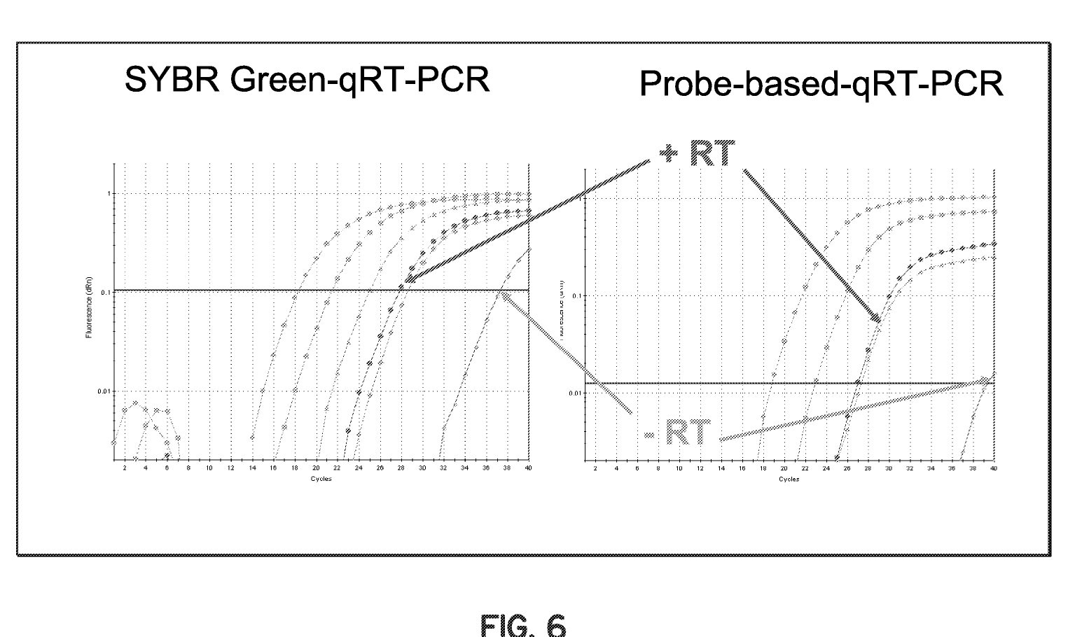

As shown in FIG. 6, dilutions of cytoplasmic lysates from cultured cells were used successfully in SYBR® Green and probe-based qRT-PCT to measure gene expression. No clean up or DNA removal step was necessary prior to the qRT-PCR reaction. About 2×10⁷ HeLa cells were trypsinized, centrifuged at about 1,000×g for one minute, and rinsed with cold PBS. Pellets containing 1×10⁶ cells were processed as described. qRT-PCR was performed using Brilliant® SYBR® Green qRT-PCR Master Mix, 1-step kit (Stratagene) or ABsolute qRT-PCR Mix (ABgene) with primers specific to exon 4 of the human β-actin gene. Dilutions ranging from 100 ng (for SYBR Green assay) or 10 ng (probe-based assay) to 100 pg of human HeLa cell total RNA (standard curve) or a 1:100 dilution of unpurified cytoplasmic fraction were added to each reaction. Data were collected on a Stratagene Mx3000P™ Real Time PCR System. In FIG. 6, each data point in each line represented the amount of amplicon generated (as a measure of fluorescence, along the Y axis) at each PCR cycle (PCR cycle number across the X axis). The β-actin amplicon generated from the cytoplasmic lysate was about 200 pg (to the left of the 100 pg standard).

For monitoring siRNA-mediated silencing, HeLa cells were transfected with ±GAPD(H) siRNA using siPORT™ NeoFX™ transfection reagent (Ambion). About 540,000 HeLa cells were trypsinized, centrifuged at about 1300×g for two minutes, and rinsed with cold phosphate buffered saline (PBS). Pellets were processed as previously described. RT-PCR was performed by priming with random hexamers, reverse transcription with MMuLV, and 30 cycle PCR with primers specific to exon 13 of the human GAPD(H) gene. 100 ng of human HeLa cell total RNA (control) or a 1:10 dilution of unpurified cytoplasmic lysate (representing about 1000 cells) was added to each reaction. Products were subject to electrophoresis on a 1% (w/w) agarose gel and visualized with ethidium bromide.

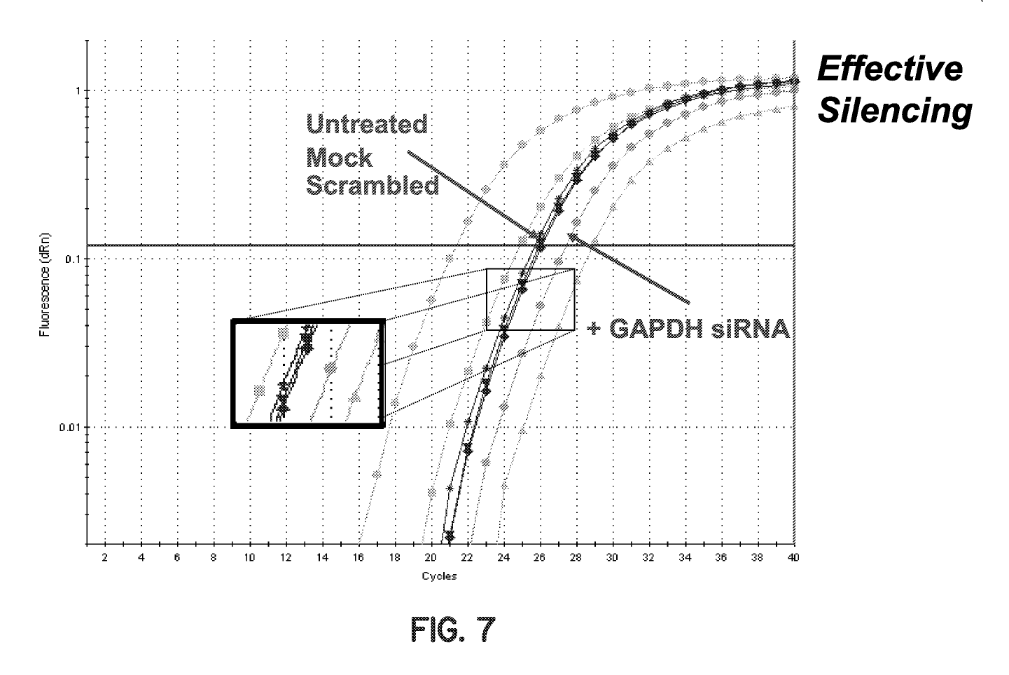

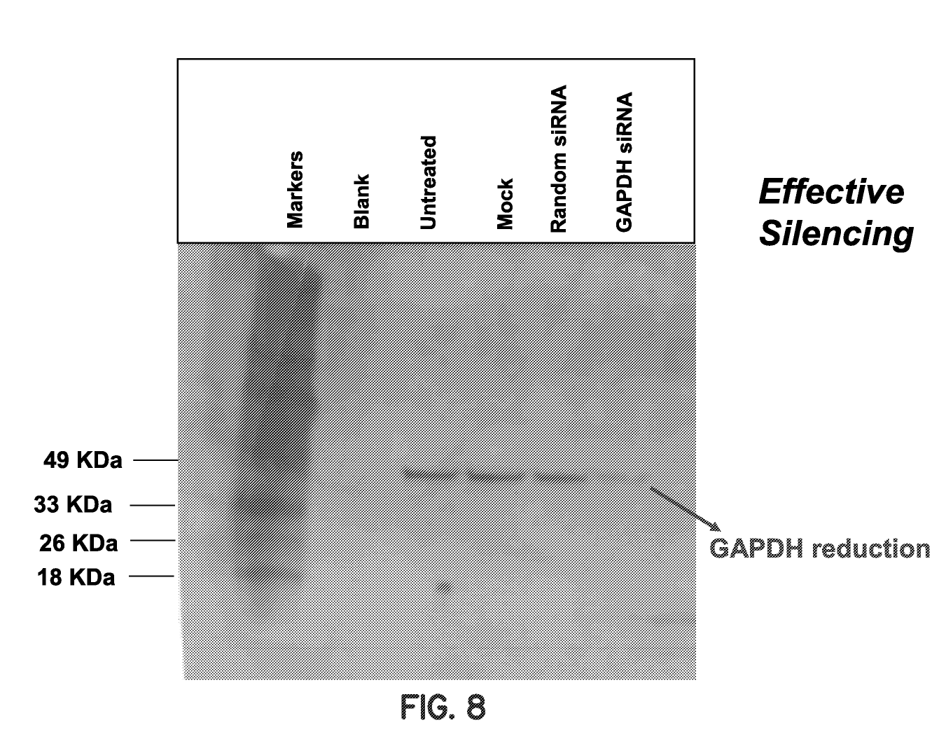

Results were obtained under each of the following conditions for 48 hours in a 24 well culture plate: no siPORT™ treatment, siPORT™ treatment, scrambled siRNA in siPORT™, and wild type siRNA in siPORT™. After 48 hours, cells were lysed using the described cytoplasmic lysis reagent. Cell debris was pelleted, and supernatant without any purification was used directly in one-step qRT-PCR. Silencing results were detected with SYBR® Green assay and confirmed by Western blots. Dilutions of the cytoplasmic lysate were used directly in qRT-PCR with GAPD(H) primers. Results are shown in FIG. 7 (RNA) and FIG. 8 (protein).

The method was also used with intra-exon RT-PCR via β-tubulin amplification in HeLa cells and Bcl2l1 amplification in mouse liver and kidney cells (data not shown). About 0.2 g of fresh mouse kidney or liver tissue was diced, placed in a homogenizer, and mixed with cytoplasmic extraction reagent as described. The samples were placed on ice for 15 minutes, mixed for 10-15 strokes or until pulverized, and centrifuged at about 16,000×g at 4° C. for ten minutes. The supernatant containing unpurified cytoplasmic lysates was collected. RT-PCR was performed by priming with random hexamers, with and without reverse transcriptase (MMuLV), and 30 cycle PCR with primers specific to exon 2 of the mouse Bcl2l1 gene.

### EXAMPLE 4

Using cytoplasmic lysate from mouse tissue, prepared as previously described, a downstream RT-PCR reaction was conducted without including the reverse transcription enzyme. This was a control for selective lysis such that the nuclei were left largely intact, as shown in FIGS. 1 and 5. In embodiments the control reaction included both RT-PCR and qRT-PCR, as shown in FIGS. 5 and 6. A product produced by the subsequent PCR reaction in the absence of reverse transcriptase indicated genomic DNA contamination. PCR reactions directed at housekeeping genes such as actin, GAPD(H), and Bcl2l1, in the presence and absence of RT, may be used to indicate selective lysis. For example, two primers that are directed to the same exon region of a gene will result in a PCR product in the presence of nuclear material, regardless of whether the sample was first subjected to an RT reaction. However, in the absence of nuclear material, a PCR product will result only following an RT reaction, as shown in FIGS. 1 and 6. Alternatively, PCR primers selected to span intron/exon junctions were used to determine whether the template was complementary DNA (cDNA) created from messenger RNA (mRNA) or genomic DNA. For example, primers selected such that one primer was directed to an intron region of a gene and the other primer directed to an exon region produced a PCR product in the presence of nuclear material, regardless of whether the sample was first subjected to an RT reaction. However, in the absence of nuclear material, no PCR product was produced under either condition (with or without prior RT reaction) (FIGS. 1 and 2). Alternatively, the lysate produced by the inventive method followed with or without subsequent RT reaction was subjected to agarose and/or acrylamide gel electrophoresis, as known to one skilled in the art (FIGS. 4 and 5).

### EXAMPLE 5

The cytoplasmic lysate generated using the described method was used to measure both mRNA and protein levels simultaneously. The cytoplasmic lysate was subjected to qRT-PCR to monitor siRNA mediated gene silencing, as well as Western blotting to confirm silencing by reduction in protein amounts. Cells grown in 24-well tissue culture plates were lysed using the described method and the lysate is used in SYBR Green-based qRT-PCR reactions.

135,000 HeLa cells (45,000 cells per well) were transfected with GAPD(H) siRNA using siPORT™ NeoFX™ Transfection Reagent (Ambion) and grown for 48 hours in a 24 well culture plate (four samples total: no siPORT™ treatment, siPORT™ treatment, scrambled GAPD(H) siRNA in siPORT™, and wild type siRNA in siPORT™). At 48 hours, after doubling approximately twice, the cells were trypsinized, centrifuged at about 1300×g for two minutes, and rinsed with cold PBS. Pellets containing about 540,000 cells were processed as described. qRT-PCR was performed using Brilliant® SYBR® Green qRT-PCR Master Mix, 1-step kit (Stratagene) with primers specific to exon 13 of the human GAPD(H) gene. Dilutions ranging from 10 ng to 100 pg of human HeLa cell total RNA (standard curve) or a 1:10,000 dilution of cytoplasmic fraction, representing about five cells, were added to each reaction. Data were collected on a Stratagene Mx3000P™ Real Time PCR System. In FIG. 7, each data point in each line represented the amount of amplicon generated (as a measure of fluorescence, along the Y axis) at each PCR cycle (PCR cycle number across the X axis). Treatment with no siPORT™ (untreated), siPORT™ (mock), and the scrambled GAPD(H) siRNA in siPORT™ (scrambled) sample lysates had similar amount of GAPD(H) RNA (about 0.6 ng), and wild type siRNA in the siPORT™ (GAPD(H)) sample had 0.2 ng of GAPD(H) RNA, indicating about 70% reduction in target RNA via silencing.

The cytoplasmic lysate was also used directly on polyacrylamide gels for the analysis of proteins, for example to monitor reduction in target protein level, via Western blotting. Results are shown in FIG. 8. All cytoplasmic lysates were separated by electrophoresis on denaturing polyacrylamide gels, electrophoretically transferred to a PVDF membrane, and the transferred protein was analyzed using antibodies against GAPD(H) and secondary antibody conjugated to horseradish peroxidase (HRP) and detected using a chemiluminescent substrate. Untreated, mock, and scrambled samples showed a distinct GAPD(H) protein band, which was reduced in the GAPD(H) siRNA sample.

### EXAMPLE 6

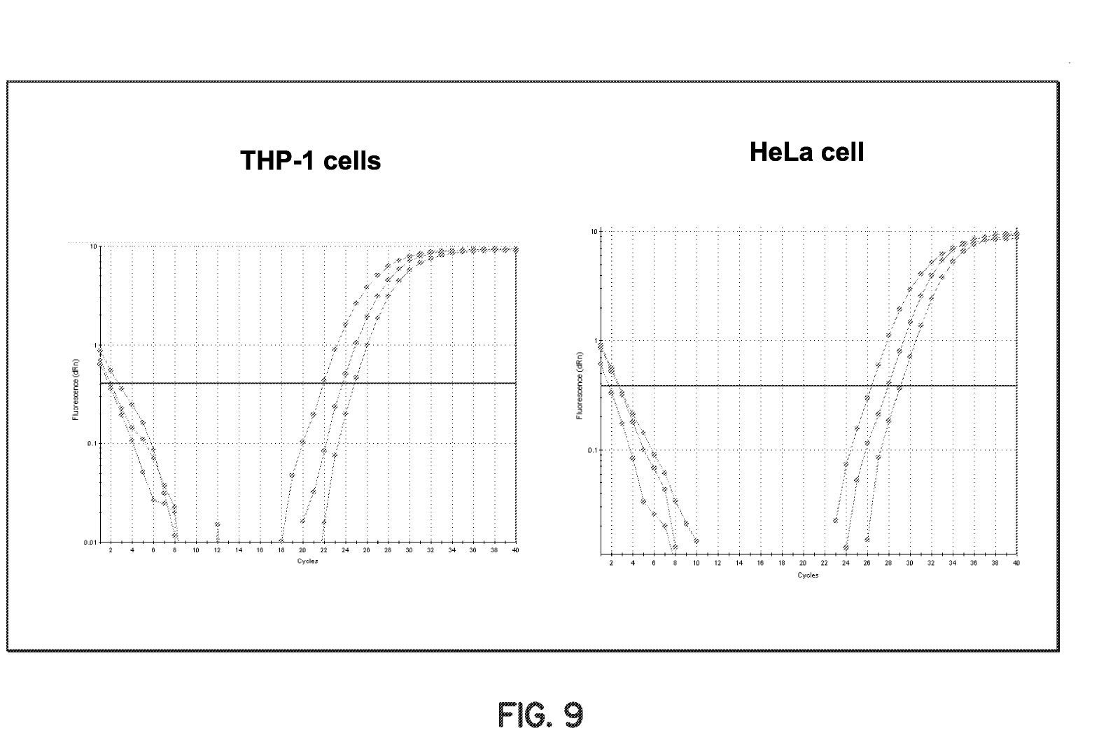

The cytoplasmic lysate generated using the described inventive method was used to assay for microRNA (miRNA) levels. Cytoplasmic lysates diluted in either water or qRT-PCR buffer were used to assay mir16 microRNA via probe-based microRNA assay (ABI). Results are shown in FIG. 9 for cytoplasmic lysate from THP-1 cells (left panel of FIG. 9) and HeLa cells (right panel of FIG. 9), using Taqman™ assays for mir 16RNA and two-step RT-PCR reactions.

About 1×10⁶ THP-1 cells (left panel) or HeLa cells (right panel) were lysed as described. The cytoplasmic lysate was used directly in qRT-PCR reactions using a two-step miRNA detection kit (ABI) for monitoring mi16RNA. For THP-1 cells, dilutions of cytoplasmic lysate used were 1:50, 1:250, and 1:750. For HeLa cells, dilutions of cytoplasmic lysate used were 1:100, 1:500, and 1:1500. Data were collected on a Stratagene Mx3000P™ Real Time PCR System. Each data point in each line represented the amount of amplicon generated (as a measure of fluorescence, along the Y axis) at each PCR cycle (PCR cycle number across the X axis). Increasing amounts of lysate resulted in earlier detection of amplified mi16RNA product.

Cytoplasmic lysate or the RNA purified from the cytoplasmic lysate was used in Northern blot assays to detect, for example mir16 microRNA, using methods known by one skilled in the art.

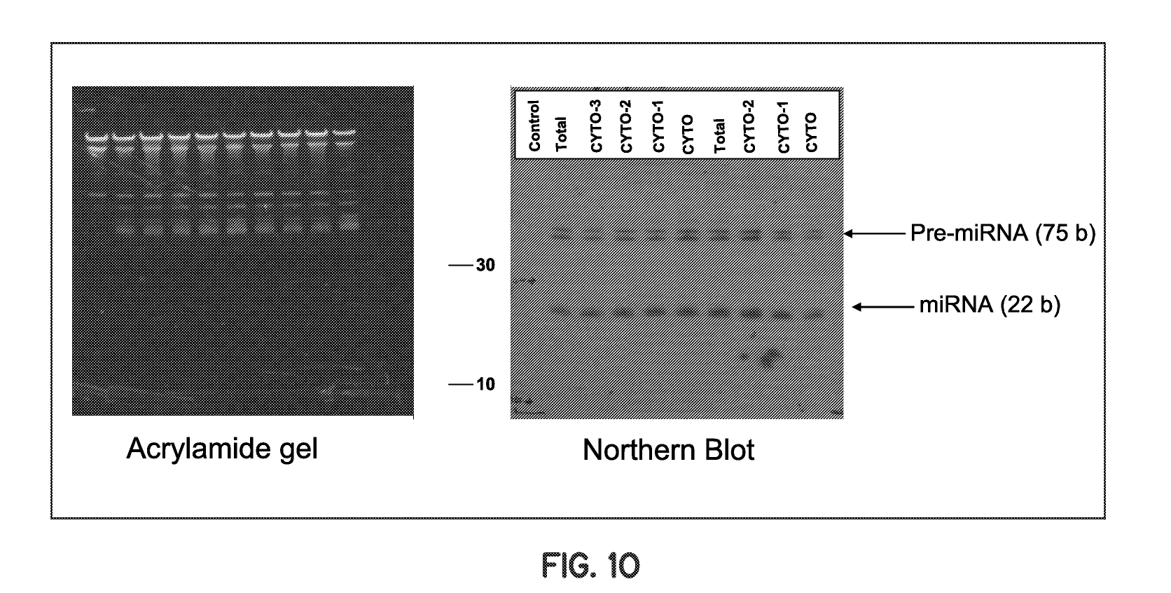

About 4×10⁶ HeLa cells were lysed via cytoplasmic cell lysis or total cell lysis. Total RNA was purified from both the cytoplasmic lysate and total cell lysate using silica-based columns. About 2 μg RNA was subjected to electrophoresis on a 15% acrylamide-urea gel, blotted onto a Biodyne membrane, and probed with ³³P labeled mir16 probe. The results are shown in FIG. 10. The panel to the left shows the RNA bands (ribosomal RNA (rRNA), messenger RNA (mRNA), and transfer RNA (tRNA)) on a acrylamide gel. The panel on the right shows hybridization signals for mi16RNA (pre-miRNA and mature miRNA).

### EXAMPLE 7

Cytoplasmic lysate generated using the described inventive method was used in procedures to purify RNA by segregating it from protein present in the cell lysate. The RNA was bound to a surface, for example, a glass fiber. Due to the absence of DNA in the lysate, methods to purify the RNA from the lysate can be readily automated. The procedure may be used for purification of RNA species such as mRNA, microRNA, tRNA, rRNA, etc., as shown in FIG. 10.

The method described herein, by which the cytoplasm was lysed while the nuclear membrane was not lysed and hence the nuclear contents were intact, allowed access to cytoplasmic RNA without interference by nuclear contents. Such cytoplasmic lysates were used directly in RT-PCR and/or qRT-PCR, with only fully processed RNA for gene expression measured, thus measurement of gene expression was not subject to any bias from purification methods. The method resulted in improved correlation among concentrations of miRNA, mRNA, and protein. The reduced sample viscosity made the method more amenable for high throughput screening and automated assays.

It should be understood that the embodiments and examples described are only illustrative and are not limiting in any way. Therefore, various changes, modifications or alterations to these embodiments may be made or resorted to without departing from the spirit of the invention and the scope of the following claims.

What is claimed is:

1. A cellular RNA isolation method comprising:
(a) treating at least one nucleated cell with a composition comprising detergent(s), buffer(s), chelator(s), reducing agent(s), salt(s), and RNase inhibitor(s) to result in a cytoplasmic lysate containing RNA and an intact nucleus,
(b) thereafter separating the cytoplasmic lysate from the nucleus, and
(c) using the cytoplasmic lysate for RNA analysis without further purifying the RNA.

2. The method of claim 1 wherein the RNA is used for at least one of RT-PCR, qRT-PCR, ribonuclease protection assays (RPA), Northern blots, primer extension assays, mRNA quantitation assays, monitoring siRNA-mediated knock-down of gene expression, profiling ribosomal RNA (rRNA) signatures, monitoring micro RNA (miRNA), and studying RNA-protein complexes.

3. The method of claim 1 wherein the cell is selected from at least one of isolated cells, cells in culture, blood, or yeast.

4. The method of claim 1 wherein the cell is from a tissue and the tissue is treated with the composition.

5. The method of claim 4 wherein the tissue is fresh or frozen.

6. The method of claim 1 wherein steps (a) and (b) take less than about 20 minutes.

7. A kit comprising
a reagent comprising at least one of each of a detergent, buffer, chelator, reducing agent, salt, and RNase inhibitor, each at a concentration to result in a cytoplasmic lysate and intact nucleus when the reagent is mixed with cells and/or tissues, and
instructions for use to result in a cytoplasmic lysate and intact nucleus.

8. The kit of claim 7 wherein the detergent is selected from the group consisting of an ionic detergent, a non-ionic detergent, a zwitterionic detergent, and combinations thereof.

9. The kit of claim 7 wherein the chelator is ethylenediaminetetraacetic acid (EDTA).

10. The kit of claim 7 wherein the buffer is selected from the group consisting of N-2-hydroxyethylpiperazine-N′-2-ethanesulfonic acid (HEPES), phosphate buffer, Tris-HCl, and combinations thereof.

11. The kit of claim 7 wherein the reducing agent is selected from the group consisting of tris-carboxyethyl-phosphine-HCl (TCEP-HCl), dithiothreitol (DTT), β-mercaptoethanol, and combinations thereof.

12. The kit of claim 7 where the salt is selected from the group consisting of KCl, NaCl, and combinations thereof.

13. The kit of claim 7 further including reagents for quantitative reverse transcription-polymerase chain reaction (qRT-PCR).

14. The kit of claim 7 further including reagents for reverse transcription-polymerase chain reaction (RT-PCR).

15. The kit of claim 7 further including reagents for high throughput RNA assay.

16. The kit of claim 7 wherein the RNase inhibitor is segregated from the detergent, buffer, chelator, reducing agent, and salt.

17. A method for cell component analysis comprising
(a) treating at least one nucleated cell with a composition comprising detergent(s), buffer(s), chelator(s), reducing agent(s), salt(s), and RNase inhibitor(s) to result in a cytoplasmic lysate containing RNA and protein, and an intact nucleus,
(b) thereafter separating the cytoplasmic lysate from the nucleus, and
(c) using the cytoplasmic lysate for at least one of RNA analysis without further purifying the RNA, protein analysis, or RNA-protein analysis.

18. The method of claim 17 wherein protein analysis comprises at least one of polyacrylamide gel electrophoresis, Western blots, qualitative assay, or quantitative assay.

19. The method of claim 17 wherein RNA-protein analysis comprises at least one of correlating RNA-protein levels, correlating microRNA, messenger RNA, and protein levels, or monitoring RNA-protein interactions.

20. The method of claim 17 wherein RNA analysis comprises at least one of RT-PCR, qRT-PCR, ribonuclease protection assays (RPA), Northern blots, primer extension assays, mRNA quantitation assays, monitoring siRNA-mediated knock-down of gene expression, profiling ribosomal RNA (rRNA) signatures, or monitoring micro RNA (miRNA).
